(19) United States
(12) Patent Application Publication
(10) Pub. No.: US 2018/0056013 A1
(43) Pub. Date: Mar. 1, 2018

(54) System, Method, and Apparatus for Delivery Through Inhalation

(71) Applicant: Kevin Knowles, Tampa, FL (US)

(72) Inventor: Kevin Knowles, Tampa, FL (US)

(21) Appl. No.: 15/620,244

(22) Filed: Jun. 12, 2017

Related U.S. Application Data
(60) Provisional application No. 62/378,657, filed on Aug. 23, 2016.

(51) Int. Cl. A61M 15/00; A61M 16/00; A61M 16/14; A61B 5/00

(52) U.S. Cl. CPC A61M 15/0005; A61M 15/003; A61M 16/0066; A61M 16/14; A61M 2209/088; A61M 15/002; A61M 2205/3303; A61M 2205/3592; A61B 5/4809

(57) ABSTRACT

A system for delivery of a material through inhalation includes a delivery cartridge having contained there within a breakable container. The breakable container has there within a payload such as a scent-bearing liquid (e.g. essential oil, perfume, etc.), medicinal liquid, etc. A device for flowing air/gases (e.g., a fan) forces air/gases through the delivery cartridge wherein after the breakable container is broken, the payload mixes with the air/gases for delivery to a user by way of inhalation.

### CROSS-REFERENCE TO RELATED APPLICATION

This application claims the benefit of U.S. provisional application No. 62/378,657 filed on Aug. 23, 2016, the disclosure of which is incorporated by reference. This application is related to U.S. non-provisional application titled “Delivery Cartridge,” filed even date.

### FIELD

This invention relates somewhat to the field of health and more particularly to a system for delivering a material by way of inhalation, perhaps for aromatherapy or medicinal reasons.

### BACKGROUND

Aromatherapy uses the natural oils extracted from flowers, stems, bark, leaves, roots etc. These essential oils are used to improve psychological and physical well-being of a person. Aromas from essential oils are believed to stimulate several brain functions. Other homeopathic benefits are also asserted by the use of essential oils.

The health benefits of aromatherapy have long been studied, including correlations between breathing aromas from essential oils and improving memory retention. This is supported by “Remembering After We Sleep Could be a Smell Away,” by Lisa M. P. Munoz, published in The Journal of Cognitive Neuroscience, Feb. 4, 2014. Essential oils are known to have a variety of therapeutic and medicinal benefits. For example, peppermint has energizing effect when inhaled, rosemary has cognitive enhancing properties, and lavender has calming and soothing properties.

For example, stimuli-based learning requires a student to be exposed to the same scent twice during the learning process. The student's first exposure of the scent is during the active learning process (i.e. studying) and the student's second exposure to the scent is during the Slow Wave Sleep (SWS) stage for declarative memory consolidation and during the REM sleep for non-declarative or procedural memory consolidation.

Medical research has found that the olfactory system provides an opportunity to delivery medicines and therapeutics; and the olfactory system can bypass the traditional methods of delivery (i.e. intravenous, oral, dermal, injection, surgical, etc.) that can sometimes be less effective, unpleasant, costly, or even harmful. Areas targeted by aromatherapy are, for example, pain management, cognitive disorders such as Down's, ADD, ADHD, autism, dementia, Alzheimer's, cancer treatment, and others, including sports medicine and sports enhancement. Further, with the advent of virtual reality, there is a need to include the olfactory senses in the experiences of the user.

The methods and techniques used in the past to deliver the essential oils (and other scents) to a user are less than optimal. Prior delivery systems typically included a night-stand device into which a user would pour a liquid (e.g. essential oil) from a bottle. In such systems, those who are not being treated are also affected by such aromas or essential oil delivery techniques. The act of pouring the essential oil into these systems often causes spillage and the essential oils often contaminate the user's clothing and skin. Some essential oils lead to allergic reactions, skin irritation, and have been known to cause health issues and illness. Further, these systems have a reservoir for holding an amount of the liquid. Once the reservoir is filled, use of a different essential oil will result in a mixed aroma that may not lead to the desired outcome. In addition, as the essential oils adhere to the reservoir even after depletion, small amounts of the prior essential oil remain in the reservoir and mix with a different essential oil when the different essential oil is then poured into the reservoir.

For at least the above reasons stated, there is need for improvements in the technologies, systems, and methods currently available for assisting in learning new material, for utilizing essential oil applications (aromatherapy), for administering medicinal agents for chronic and acute disorders, diseases, syndromes, etc. in both animals and humans, for enhancing the experience of virtual reality, for enhancing the performance of athletes, and other potential uses when the olfactory system is involved.

Further, as medical delta-9-tetrahydrocannabinol (THC) begins to be used for various illnesses, TCH dissolved in lipids or alcohols may be inhaled to extract the medicinal values of the phytocannabinoid family.

What is needed is a system for personal delivery of a material (e.g. a scent, perfume, medicine) by way of inhalation.

### SUMMARY

In one embodiment, a system for delivery of a material for inhalation is disclosed including a delivery cartridge having contained there within a breakable container. The breakable container has there within a payload (e.g. a liquid, medicine, essential oil). A device for flowing air (e.g., a fan) forces air/gases through the delivery cartridge wherein after the breakable container is broken, the payload mixes with the air for delivery to a user by way if inhalation.

In another embodiment, a method of delivering a material through inhalation is disclosed including flowing air through a delivery cartridge after having broken a breakable container within the delivery cartridge, thereby releasing a material from the breakable container. The breakable container has there within the material (e.g. a liquid such as an essential oil, medicinal liquid, perfume). The method includes flowing air/gases towards a user for delivery of the material to the user by inhalation by the user, for example, for use in aromatherapy.

In another embodiment, an inhalation delivery system is disclosed including a housing for wearing by a user and a delivery cartridge. The delivery cartridge contains there within a breakable container. The breakable container has there within a payload such as an essential oil, perfume, medicinal liquid, etc. A receiver holds the delivery cartridge within the enclosure. A fan is interfaced to the receiver for flowing air/gases through the delivery cartridge, the air/gases coming from outside of the housing through one or more vents. The fan channels the air/gases mixed with the material through a grill for emitting the air/gases containing the material towards a user for inhalation. After the breakable container is broken, the material mixes with the air/gases as the air/gases flows through the delivery cartridge.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a perspective view of an embodiment of a device for delivering a material for inhalation.

FIG. 2 illustrates a second perspective view of the embodiment of a device for delivering a material for inhalation.

FIG. 3 illustrates a cut-away perspective view of the embodiment of a device for delivering a material for inhalation.

FIG. 4 illustrates an exploded view of a portion of the embodiment of a device for delivering a material for inhalation.

FIGS. 5 and 5A illustrate plan views of a delivery cartridge of the device for delivering a material for inhalation.

FIGS. 5B, 5C, 5D, 5E, 5F, 5G, 5H, 5I, 5J, 5K, 5L, 5M, 5N, 5P, 5Q, and 5R illustrate plan views of end caps of the delivery cartridge of the device for delivering a material for inhalation.

FIGS. 6A, 6B, 6C, and 6D illustrate elevational views of end caps of the delivery cartridge of the device for delivering a material for inhalation.

FIG. 7 illustrates a schematic view of a discrete device for delivery of a material by inhalation.

FIG. 8 illustrates a schematic view of a processor-based device for delivery of a material by inhalation.

FIG. 9 illustrates a first program flow of the processor-based device for delivery of a material by inhalation.

FIG. 10 illustrates a first program flow of the processor-based device for delivery of a material by inhalation.

FIG. 11 illustrates a perspective view of a fitness tracking device.

FIG. 12 illustrates a perspective view of the device for delivery of a material by inhalation that is integrated into a wristband.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

The embodiments shown are by examples of those anticipated for the personal delivery of a scent (e.g. aromatherapy), limited for brevity and clarity purposes only. It is fully anticipated that the delivery technology disclosed herein be integrated into any number of devices, either worn by the user or located in the vicinity of the user. These devices are, for example, neckwear (as disclosed, though in any physical format), eyewear (e.g. glasses), headwear (e.g. hats, straps, masks, headbands), ear wear, table-top or nightstand devices, automobile visor-mounted devices, wristbands (as disclosed, though in any physical format), waistbands, chest bands, clothing, helmets (e.g. football helmets), sport masks or faceguards, headsets, handheld devices, rings, gloves, armbands, extra-nasal devices, intranasal devices, cannula devices, facial masks covering the mouth and/or nose, necklaces, pendants, lanyards, collars, etc.

In an exemplary use of the device for delivery of a material by inhalation, the device for delivery of a material by inhalation is worn by or placed near a user. The device for delivery of a material by inhalation is first energized during the studying process (encoding). Later, the device for delivery of a material by inhalation is energized a second time during sleep while the brain is within the memory consolidation process. The device for delivery of a material by inhalation is worn during two of the three major sub-processes of memory function. Specifically, during the encoding process (i.e. studying) and during the memory consolidation (reactivation) process which occurs during deep sleep. Slow Wave Sleep or SWS is the period of sleep for declarative memory consolidation, Rapid Eye Movement or REM is the period of sleep for non-declarative or procedural memory consolidation. U.S. Pat. No. 8,382,484 to Wetmore, et al., describes the sleep cycles and memory consolidation. This patent is hereby incorporated by reference.

It is anticipated that the device for delivery of a material by inhalation described herein be used to help a hyperactive child learn. For example, the device for delivery of a material by inhalation (in this example, aromatherapy) described herein is used to deliver a specific aromatherapy known to calm the child, relax the child, help the child concentrate, etc. Further, the device for delivery of a material by inhalation described herein is used to deliver a specific aromatherapy during learning (e.g. in the class room), then during when the child sleeps. For the device for delivery of a material by inhalation to be of maximum effect, the device for delivery of a material by inhalation is used during sleep. The device for delivery of a material by inhalation is configured to not interfere with the child's sleep, for example, by delaying emission of the aromatherapy and remaining quiet while the child falls asleep.

The educational benefits of the device for delivery of a material by inhalation are anticipated to help with declarative (cognitive) learning as well as non-declarative or procedural learning. Various applications for education are anticipated, for example, learning a foreign language, learning new course material, preparing for exams, learning to play a musical instrument, learning from flash cards, learning sports playbook, learning sales material, learning technical information, and other.

In some embodiments, the device for delivery of a material by inhalation is anticipated to be used to enhance the performance of an athlete while training or while performing. For example, a weightlifter uses the device for delivery of a material by inhalation to deliver aromatherapy while performing a difficult lifting exercise or a sprinter to uses the device for delivery of a material by inhalation to deliver aromatherapy at the start of a race.

As in some embodiments, the device for delivery of a material by inhalation emits smells, it is anticipated that versions of the device for delivery of a material by inhalation be used in emitting perfume scents, providing long-lasting scents to the wearer.

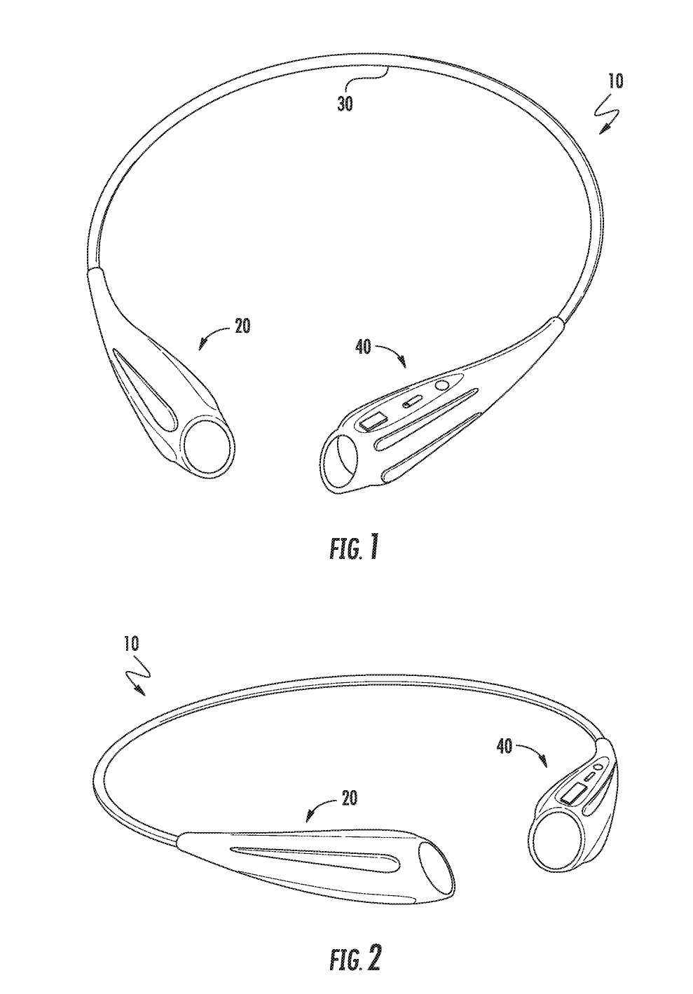

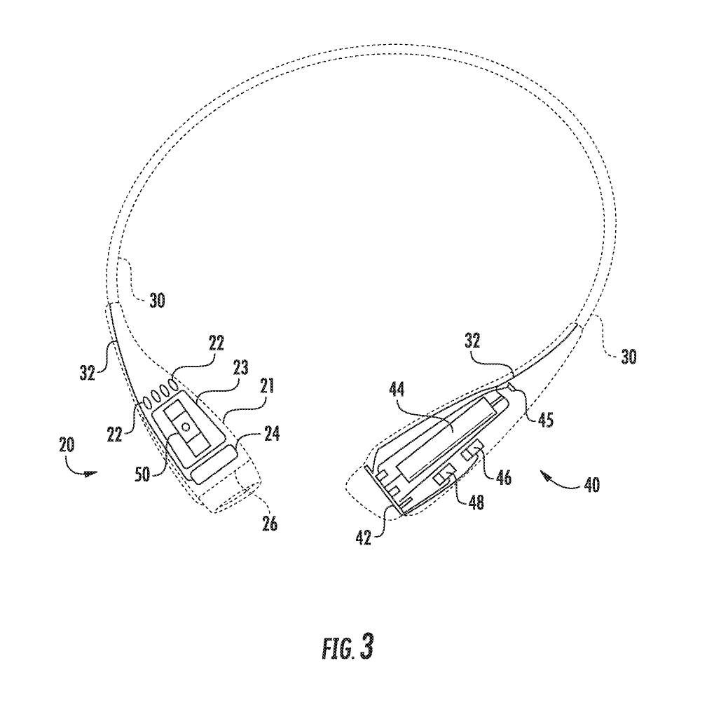

Referring to FIGS. 1-4, views of an embodiment of a device for delivery of a material by inhalation 10 are shown. It has been explained that various forms of aromatherapy are desired or found beneficial to be provided during sleep. Further, other uses for the device for delivery of a material by inhalation 10 have been discusses including, but not limited to, delivery of medicines, perfumes, and other odors and/or odorless materials. Therefore, in at least one embodiment of the instant invention, the device for delivery of a material by inhalation 10 is worn on a person. In the examples shown in FIGS. 1-4, the device for delivery of a material by inhalation is worn around the user's neck (not shown in use for clarity reasons). Further, as certain individuals enjoy perfumes, but spraying perfume on one's body and clothing does not provide a long-term odor, lasting only several hours, and some individuals are allergic to perfumes, the device for delivery of a material by inhalation 10 worn around the user's neck provides constant delivery of perfume without the perfume contacting the user's body or clothing.

The device for delivery of a material by inhalation 10 utilizes a delivery cartridge 50 that contains there within the material (e.g. essential oils, perfume, THC compounds) in liquid, gaseous, or solid/powder form, therefore eliminating the need for a user to pour such liquids/powders into a reservoir. Using the delivery cartridge 50 has several advantages including, but not limited to, the ability to instantaneously change aromas by swapping delivery cartridges 50, the ability to install the material (e.g. liquid) without the risk of spilling, staining, or human contact with the liquid, the ability to maintain multiple types of scent-bearing liquids without exposing the stored scent-bearing liquid (e.g. essential oils) to air, the ability to mix multiple materials at the time of use (e.g. a powder and a liquid, two liquids), etc.

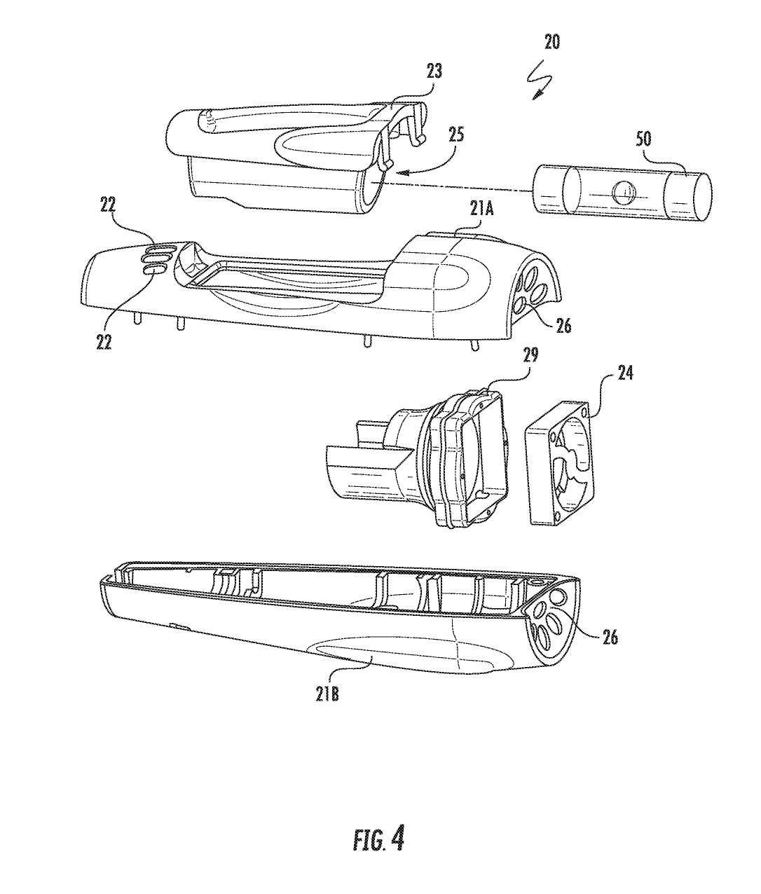

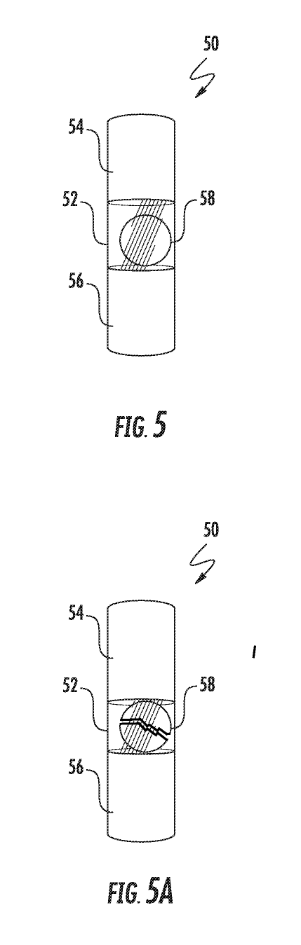

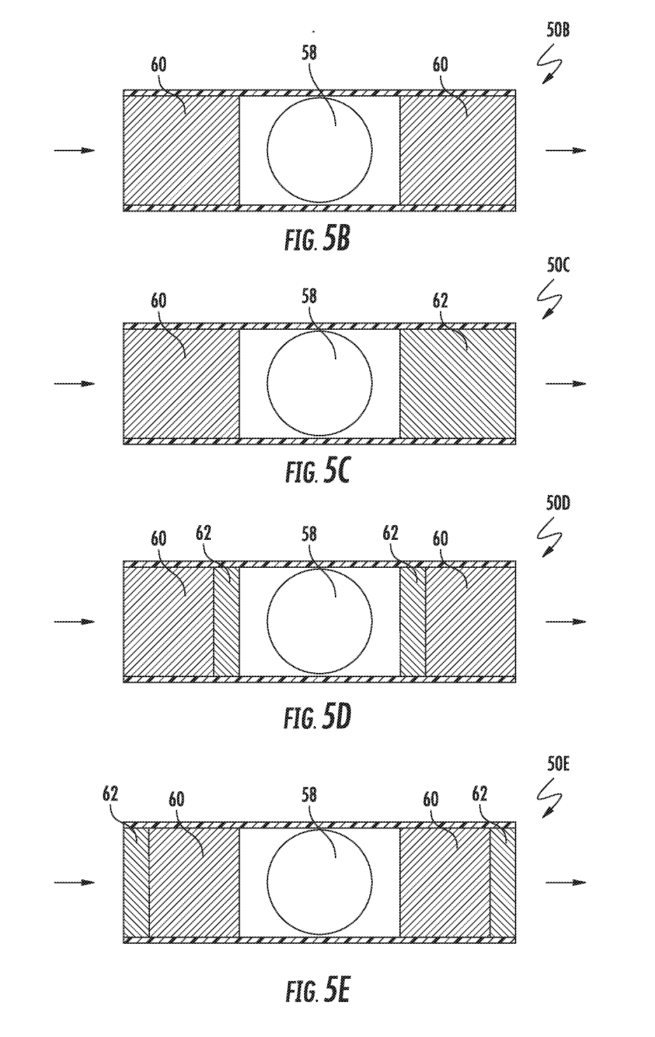

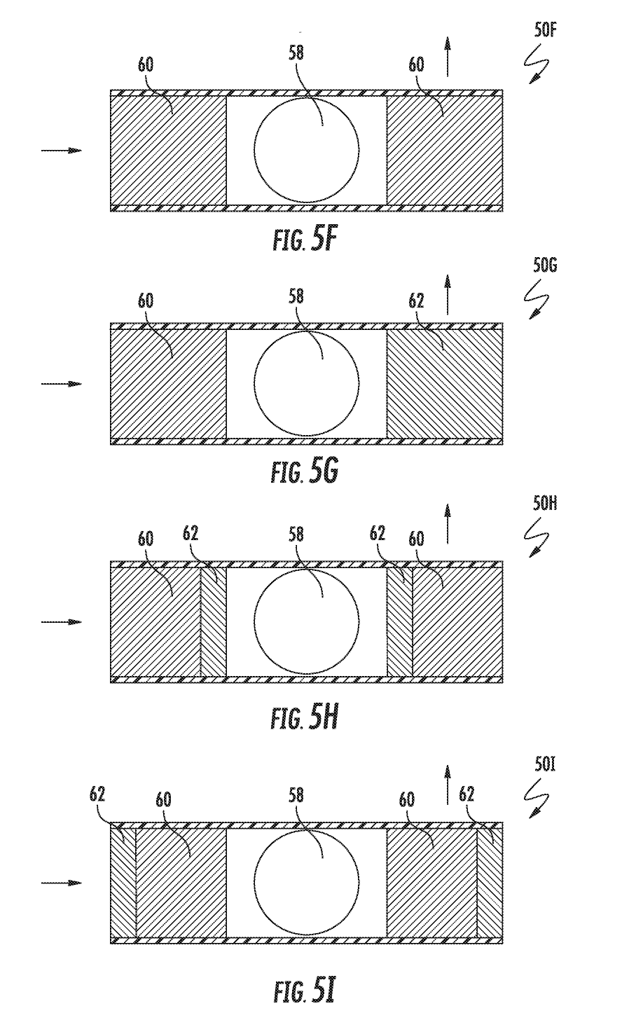

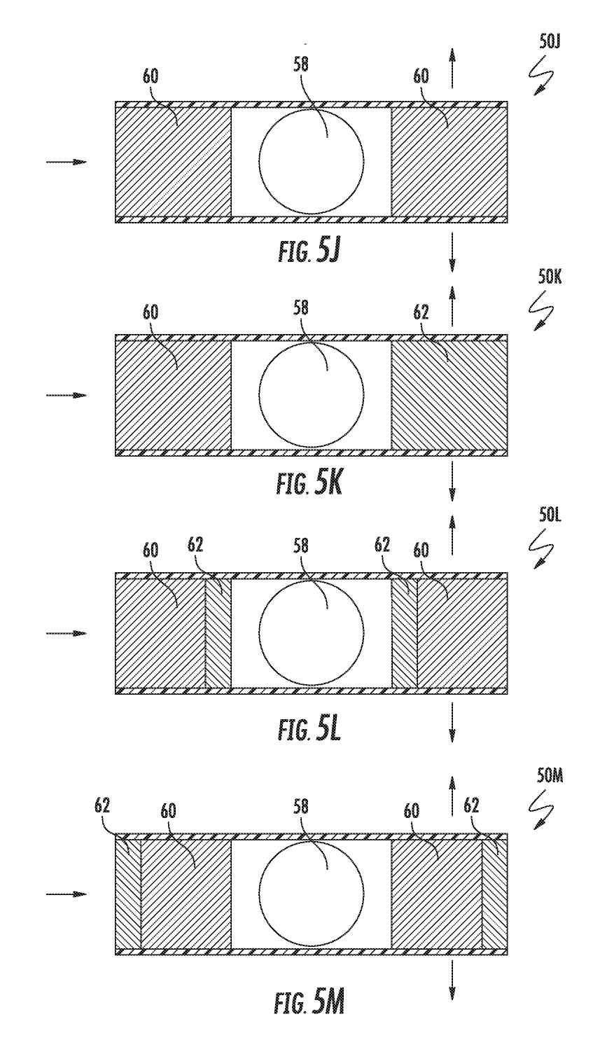

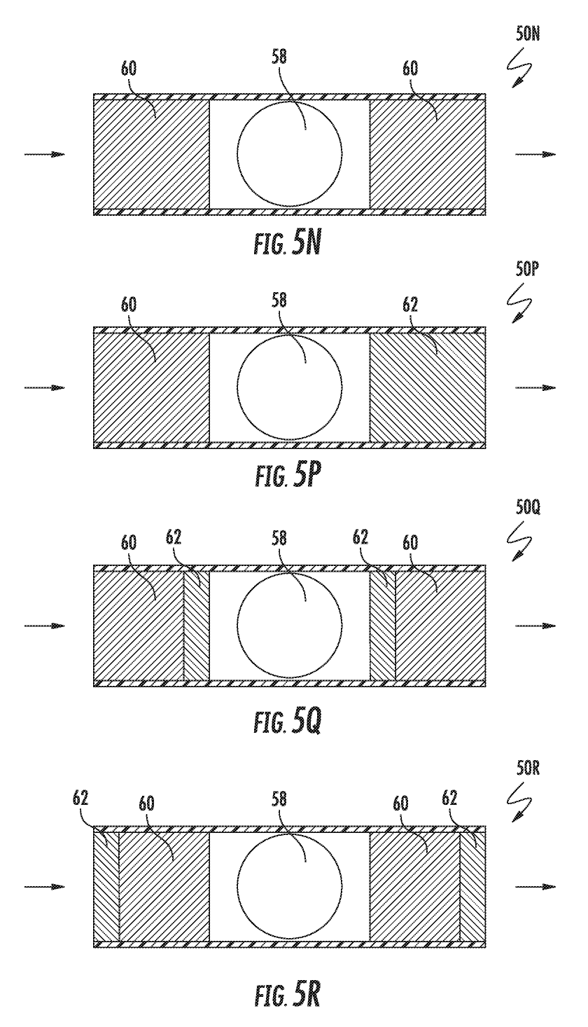

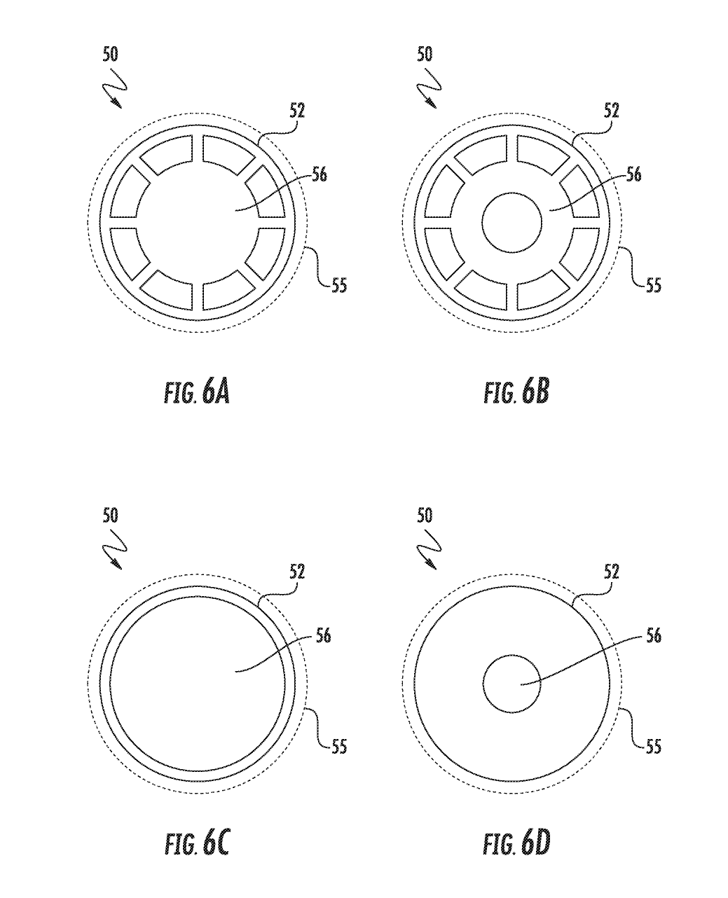

To provide the necessary components in a balanced device for delivery of a material by inhalation 10, one embodiment of the device for delivery of a material by inhalation 10 of FIGS. 1-4 has an enclosure with two end portions 20/40 connected by a connecting portion 30. The first end portion 20 houses a fan 24 held behind a grill 26. The first end portion 20 accepts the delivery cartridge 50 (see FIGS. 5 and 6) and the fan moves air in from vents 22, through the delivery cartridge 50 and out of the grill 26 in a direction towards a person who is wearing the device for delivery of a material by inhalation 10. It is equally anticipated to place the fan on the other side of the delivery cartridge 50 to push air through the delivery cartridge 50 instead of pulling air through the delivery cartridge 50.

The delivery cartridge 50 contains the material(s) (e.g. liquid essential oils, perfume, THC). The delivery cartridge 50 is removably held within the first end portion 20 within or beneath a cover 23 or receiver. One or more breakable containers 58 within the delivery cartridge contain the material(s) until it is time to use the delivery cartridge 58, at which time the one or more breakable containers 58 are broken, releasing the material(s) within the delivery cartridge 50. The breakable container 58 within the delivery cartridge 50 is broken by the user by, for example, squeezing, to release the material(s) (e.g. aromatherapy liquid, essential oils, perfume, THC, etc.), placed into the first end portion (or into the receiver), and then the cover 23 is placed over the delivery cartridge 50 to maintain the location of the delivery cartridge 50 and maintain proper air flow through the delivery cartridge 50. The fan receives power through wires 32 that pass through the connecting portion 30 to a control circuit 42 within the second end portion 40. For example, the wires 32 pass through a hollow area of the connecting portion 30 or the wires 32 are molded into the connecting portion 30.

The second end portion 40 contains a power source 44 (e.g. a primary battery, rechargeable battery, etc.). In some embodiments, the power source 44 is rechargeable and power for recharging is provided through a connector 45 (e.g. micro USB connector) and connected to the control circuit 42 which has charge controls for charging the power source 44 and for conditioning power as needed. It is also anticipated that power for charging be provided through any known wired or wireless connection system, including, but not limited to, two or more contacts, any type of connector, induction-coupled charging, light-powered charging, etc. The second end portion has one or more switches 46/48 for controlling operation of the fan 24 by way of the control circuit 42.

In FIG. 4, an exemplary first end portion 20 of the device for delivery of a material by inhalation 10 is shown. Although any structural system is anticipated, the exemplary first end portion 20 includes a cover 23 that has a cavity 25 into which the delivery cartridge 50 is inserted. The cover 23 snaps into the top section 21A of the exemplary first end portion 20. A fan shroud 29 channels air flow from the cavity 25, through the fan 24, and out of the grill 26. A bottom section 21B seals the fan 24 and fan shroud 29 against the top section 21A and completes the enclosure of the exemplary first end portion 20. Air pulled into the exemplary first end portion 20 enters through vents 22, passes through the delivery cartridge 50, then out of the grill 26 in a direction of the user.

Referring to FIGS. 5 and 5A, plan views of a delivery cartridge 50 of the device for delivery of a material by inhalation 10 are shown. Many existing/prior aromatherapy devices require the user to pour a liquid (e.g. an essential oil) into a reservoir before the scent or aromatherapy is delivered. There are many problems with the prior ways to dispense this liquid, including; the potential for spilling the liquid which may result in stains to clothing, carpet, etc., getting the liquid on one's body/hands; further, many such liquids evaporate and/or degrade when exposed to the air. For example, essential oils are recommended to be stored in a glass or steel vessel that is air-tight. Upon opening of a bottle of such essential oils, air enters the bottle and starts the degradation process immediately. Another issue with placing these liquids in a reservoir is limited ability to change aromas/scents. For example, if a user pours 3 ounces of a first liquid into the reservoir, the full three ounces must be dispensed or emitted before adding a different liquid as it is often undesirable to mix liquids as mixing of certain aromas/scents produces a combined aroma/scent that is often not beneficial or not desired.

To eliminate the problems of having a reservoir that is filled with, for example, a scent-bearing liquid by the user, the device for delivery of a material by inhalation 10 receives and utilizes a delivery cartridge 50 that contains one or more breakable containers 58, each having there within one or more material(s) in liquid, gaseous, or solid/powder form. Each end of the delivery cartridge 50 has an end cap 54/56 or other mechanism to contain the breakable containers 58 and to absorb the material(s) to limit leakage and maximize delivery through inhalation. After the breakable container(s) 58 is/are broken, the material(s) is/are optionally combined and absorbed into the material of the end caps 54/56. As the fan 24 moves air through the delivery cartridge 50, molecules of the material(s) (e.g. aromatherapy liquid, perfume, medicine) are drawn from the material of the end caps 54/56 and delivered for inhalation by the user through the air.

The delivery cartridge 50 has a cover 52 that contains the breakable container(s) 58. Although not required, it is preferred that the cover 52 be non-porous so that the payload does not come into contact with the user after the breakable container(s) 58 is/are broken. Once the material(s) that is sequestered within the breakable container(s) 58 is/are released into the cartridge, it is preferred that the liquid is not allowed to penetrate, permeate or saturate the cover 52. Therefore, it is preferred that at least one layer of the cover 52 be non-porous so as to contain material(s) that are in liquid form.

The breakable container(s) 58 are anticipated to encapsulate one or more material(s) in liquid, gaseous, or solid form, providing protection for the material during storage, transport, etc. This allows selective release of the material upon physical destruction or breakage of the breakable container(s) 58. Typically, the breakable container(s) 58 include an outer cover or shell surrounding a central payload region that includes the material in liquid, gaseous, or solid/powder form. Exemplary materials forming the encapsulation of the breakable container(s) include cellulose, acetate, polyvinyl acetate, polyethylene, polypropylene, sepiolite, menthol crystals, rock salt crystals, crystalline sugar, cyclodextran, corundum, quartz crystals, metal (e.g., thin copper or thin stainless steel), pumice, agglomerated calcium carbonate, microcrystalline cellulose particles, etc. In some embodiments, the material is a lipophobic material, though this is not a complete list as others are anticipated.

Further, in some embodiments, the cover 52 is clear or translucent so as to allow the user visibility of the breakable container(s) 58 so the user knows when the breakable container(s) 58 have been broken. In some embodiments, the cover 52 is or includes a lipophobic material (fat rejecting), as essential oils are typically lipids. In some embodiments, the cover 52 is or includes a hydrophobic material for containing a water-based liquid. Any material or combination of materials is/are anticipated for the breakable container 58 as long as the selected material contains the payload (especially liquid materials) until outside pressure causes breakage of the breakable container 58. In some embodiments, the delivery cartridge includes fingers, pins, or other projections (not shown) for aiding in breaking of the breakable container(s) 58.

Although the delivery cartridge 50 is shown with a first end cap 54, a breakable container 58, and a second end cap 56, in that order, any internal configuration is anticipated. For example, it is anticipated that there be an intermediate portion made of wicking material 60 and/or non-wicking material 62 with a breakable container 58 on each side of the intermediate portion, thereby allowing breaking of one breakable container 58 while leaving the second breakable container 58 intact for later use.

In FIG. 5, a single, intact, breakable container 58 is shown as an example. In FIG. 5A, the single breakable container 58 is shown broken after physical force was applied to break the breakable container 58. Note that in FIGS. 5 and 5A, the breakable container 58 is shown as a single sphere, though any number and/or shape of breakable containers 58 is anticipated. Further, it is fully anticipated that, in some embodiments, multiple breakable containers 58 are present having two or more different materials contained in each. Further, in some embodiments, materials from one breakable container 58 react with materials from another breakable container 58 (e.g., foaming, forcing evaporation, creating chemical reactive heat, enhancing evaporation . . . ).

Although shown in generally cylindrical form, there is no limitation of size and/or shape for the delivery cartridge. Likewise, although shown with end caps 54/56 at each end, there is no limitation as to the location, size, shape, configuration, or number of orifices for flowing of a gas (e.g. air) through the delivery cartridge.

In some embodiments, as shown in FIGS. 5B, 5C, 5D, 5E, 5F, 5G, 5H, 5I, 5J, 5K, 5L, 5M, 5N, 5P, 5Q, and 5R, the end caps 54/56 are shown including wicking material 60 and/or non-wicking material 62. The wicking material and non-wicking material absorb the payload to prevent/reduce leakage, while air flowing through such delivers the payload for inhalation by the user. In FIG. 5B, the end caps 54/56 are made of wicking material 60. In FIG. 5C, the first end cap 54 is made of wicking material 60 and the second end cap 56 is made of non-wicking material 62. In FIG. 5D, the end caps 54/56 are made of both wicking material 60 and non-wicking material 62, the non-wicking material 62 being closest to the breakable container 58. In FIG. 5E, the end caps 54/56 are made of both wicking material 60 and non-wicking material 62, the non-wicking material 62 being furthest from the breakable container 58. In FIG. 5F, the end caps 54/56 are made of wicking material 60, but in this embodiment, the air flow exits from the side of the second end cap 56. In FIG. 5G, the first end cap 54 is made of wicking material 60 and the second end cap 56 is made of non-wicking material 62; in this embodiment, the air flow exits from the side of the other second cap 56. In FIG. 5H, the end caps 54/56 are made of wicking material 60 and non-wicking material 62, the non-wicking material 62 being closest to the breakable container 58; in this embodiment, the air flow exits from the side of the other second cap 56. In FIG. 5I, the end caps 54/56 are made of wicking material 60 and non-wicking material 62, the non-wicking material 62 being furthest from the breakable container 58; in this embodiment, the air flow exits from the side of the other second cap 56. In FIG. 5J, the end caps 54/56 are made of wicking material 60, but in this embodiment, the air flow exits from two sides of the second end cap 56. In FIG. 5K, the first end cap 54 is made of wicking material 60 and the second end cap 56 is made of non-wicking material 62; in this embodiment, the air flow exits from two sides of the other second cap 56. In FIG. 5L, the end caps 54/56 are made of wicking material 60 and non-wicking material 62, the non-wicking material 62 being closest to the breakable container 58; in this embodiment, the air flow exits from two sides of the other second cap 56. In FIG. 5M, the end caps 54/56 are made of wicking material 60 and non-wicking material 62, the non-wicking material 62 being furthest from the breakable container 58; in this embodiment, the air flow exits from two sides of the other second cap 56. In FIG. 5N, the end caps 54/56 are made of wicking material 60, but in this embodiment, the air flow exits from the end and sides of the second end cap 56. In FIG. 5P, the first end cap 54 is made of wicking material 60 and the second end cap 56 is made of non-wicking material 62; in this embodiment, the air flow exits from the end and the sides of the other second cap 56. In FIG. 5Q, the end caps 54/56 are made of wicking material 60 and non-wicking material 62, the non-wicking material 62 being closest to the breakable container 58; in this embodiment, the air flow exits from the end and sides of the other second cap 56. In FIG. 5R, the end caps 54/56 are made of wicking material 60 and non-wicking material 62, the non-wicking material 62 being furthest from the breakable container 58; in this embodiment, the air flow exits from the end and sides of the other second cap 56.

Note that the above includes examples of end caps 54/56 having various configurations but the composition of the end caps 54/56 are not limited to these examples, as many other configurations are anticipated.

Referring to FIGS. 6A-6D, exemplary end caps 54/56 are shown from the end of the delivery cartridge 50. In FIG. 6A, a wagon wheel design is shown with a central orifice and several outer orifices for the flow of air and the payload/material(s) through the end caps 54/56. In FIG. 6B, a Corinthian design is shown with a larger central orifice and several smaller outer orifices for the flow of air and payload/material(s) through the end caps 54/56. In FIG. 6C, a tubular design is shown with a large central orifice for the flow of air and payload/material(s) through the end caps 54/56. In FIG. 6D, a solid design is shown without any orifices and the flow of air and payload/material(s) through passes through the material of the end caps 54/56, as the material of the end caps 54/56 is generally porous. It is anticipated that in some embodiments, both end caps 54/56 have the same cross-sectional design while in other embodiments, each end cap 54/56 has a different cross-sectional design.

Note that the above includes examples of cross-sectional designs of end caps 54/56 but the end caps 54/56 are not limited to these examples, as many other configurations are anticipated.

Although the delivery cartridge 50 is shown as being generally tubular, there is no limitation as to the shape of the delivery cartridge 50. For example, it is fully anticipated that the cross-sectional shape of the delivery cartridge 50 be octagonal, rectangular, round (as shown), irregular, etc. Further, in some embodiments, one end of the delivery cartridge 50 had a different shape or size than the other end of the delivery cartridge 50 to provide directional installation into the device for delivery of a material by inhalation 10.

As used herein, the payload (material) is any flavorful, medicinal, or aromatic substance. In some embodiments, the payload (material) is capable of altering the sensory characteristics associated with air which flows through the delivery cartridge after release of the payload from encapsulation within the breakable container 58. Exemplary sensory characteristics that can be modified by the aroma/scent liquid include taste, mouth feel, moistness, coolness/heat, and/or fragrance/aroma.

In some embodiments, as shown in FIGS. 6A-6D, the cover 52 is covered or partially covered with an outer coating 55 of a material such as paper or plastic. The outer coating 55 is useful for identification as the outer coating 55 is anticipated to be printed with an identification and/or color coded to indicate the type of material contained within the breakable container 58 (e.g. red for cinnamon, green for mint, brown for coffee).

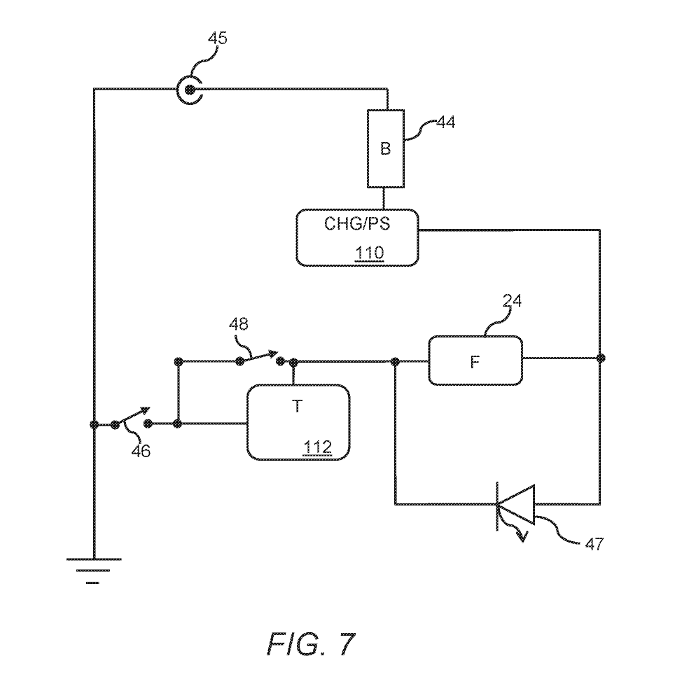

Referring to FIG. 7, a schematic view of a discrete device for delivery of a material by inhalation 10 is shown. Power is provided to the device for delivery of a material by inhalation 10 in any way known in the industry, for example, as shown, through a power jack 90 for recharging an internal power source 44 (e.g. rechargeable battery), though it is also anticipated to utilize a replaceable primary battery (e.g. an AAA battery). In the example shown in FIG. 7, the internal power source 44 (e.g. rechargeable battery) is charged by a charge control 110 that also provides regulated power to the device for delivery of a material by inhalation 10.

Although many user interfaces with the same or different configurations and operation of switches 46/48 is anticipated. In this embodiment, the first switch 48 is an on/off switch and the second switch 46 is a mode switch (e.g. in an open position “tinned”, the timer 112 operates the fan 24 for a timed start and in a closed position “delayed start”, the fan 24 goes on immediately when the first switch 48 is closed.

When the second switch 46 (mode) is open, the timer 112 starts a delay sequence upon closure of the first switch 48 (power). During the delay sequence, no power is provided to the fan 24. After the delay sequence expires, the timer 112 provides electrical power to the fan 24, thereby the fan 24 operates to move air through the delivery cartridge 50, providing, for example, aromatherapy to the user. In this way, aromatherapy is provided to the user, for example, during the memory consolidation process and not provide while the user is attempting to fall asleep.

In some embodiments, the timer 112 includes a circuit that ramps power to the fan 24 (e.g. slowly increases a speed of the fan 24 over time). Such ramping provides several advantages including hiding of the fan starting as a sudden start is more noticeable; and controlled delivery of certain materials that have a strong initial outflow of a scent or potency of a medicine (e.g. peppermint might overwhelm a user if full fan speed is applied at first, but by starting with a slow speed of the fan 24, less scent is emitted until later, when most of the peppermint has evaporated and the speed of the fan 24 is increased).

In some embodiments, the delivery cartridge 50 is encoded to indicate a mode of delivery that need be made. For example, a bar code, RFID (radio-frequency identification device), series of rings (or absence of such), colors, an end shape, a length, peaks/valleys in the cover 52 or end caps 54/56, etc. In such, the timer 112 includes circuitry to read this code to determine how and when to operate the fan 24. For example, the timer 112 includes an optical detector that is optically coupled to the cavity 25 into which the delivery cartridge 50 is inserted. Detecting a first color from the delivery cartridge 50 instructs the timer circuit 112 to initiate immediate operation of the fan 24 while detecting a second color from the delivery cartridge 50 instructs the timer circuit 112 to initiate operation of the fan 24 after a time period, while detecting a third color from the delivery cartridge 50 instructs the timer circuit 112 to initiate ramped operation of the fan 24, etc. In some embodiments, a user fan speed is provided so that the user has the ability to control a speed of the fan 24, for example during administration of a medicine or drug.

There are many timers known in the industry including electro-mechanical timers (bi-metallic, etc.), clock-movement timers, and semiconductor timers, along with many circuit configurations to achieve the same operational results; all are anticipated here within. Exemplary timers are exemplified by the industry standard 555/556 timer. In some cases, the power output of such a timer is sufficient to operate the fan 24, and/or the optional indicator 47 without the use of the relays. In some exemplary systems, relays or semiconductor relays, power transistors, or power FETs, are used to drive the fan 24 as known in the industry.

As the device for delivery of a material by inhalation 10 is often utilized during sleep, the timer 112 delays initiation of the aroma therapy until it is presumed that the user is sleeping. In some embodiments, to reduce discontinuance of sleep patterns, the timer 112 has a stepped-output function, gradually increasing the voltage/power provided to the fan 24 and gradually decreasing the voltage/power provided to the fan 24, so as to not startle the user by the sudden change in noise.

Although a fan 24 is shown for moving the air, it is fully anticipated to use any type of fan 24 or other electro-mechanical or mechanical device to move air/gases through the delivery cartridge 50, including, but not limited to, squirrel cage fans, piston/cylinder arrangements, gravity/movement translating devices, nebulizers, stored compressed air, vibrating mesh nebulizer, ultrasonic emitters, etc.

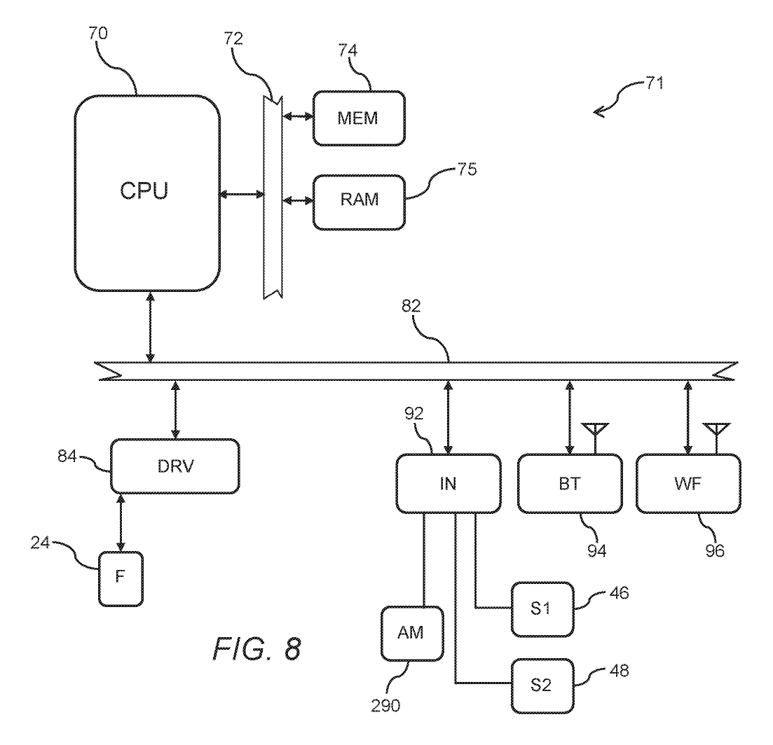

Referring to FIG. 8, a schematic view of a processor-based device for delivery of a material by inhalation 10 is shown. The exemplary processor-base 77 represents a typical processor-based system as used with the device for delivery of a material by inhalation 10, though it is known in the industry to utilize logic in place of processors and vice versa. This exemplary processor-base 77 is shown in its simplest form. Different architectures are known that accomplish similar results in a similar fashion and the device for delivery of a material by inhalation 10 is not limited in any way to any particular system architecture or implementation. In this exemplary processor base 71, a processor 70 executes or runs programs from a random-access memory 75. The programs are generally stored within a persistent memory 74 and loaded into the random-access memory 75 when needed. The processor 70 is any processor, typically a processor designed for portable devices. The persistent memory 74 and random-access memory 75 interface through, for example, a memory bus 72. The random-access memory 75 is any memory suitable for connection and operation with the selected processor 70, such as SRAM, DRAM, SDRAM, RDRAM, DDR, DDR-2, etc. The persistent memory 74 is any type, configuration, capacity of memory suitable for persistently storing data, for example, flash memory, read only memory, battery-backed memory, magnetic memory, etc. In some examples, the persistent memory 74 is removable, in the form of a memory card of appropriate format such as SD (secure digital) cards, micro SD cards, compact flash, etc.

Also connected to the processor 70 is a system bus 82 for connecting to peripheral subsystems such as output drivers 84 and input ports 92 such as control switches 46/48 and readers (not shown). The output drivers 84 receive commands from the processor 70 and control, for example, the fan 24, though other outputs such as LED are also anticipated.

In general, some portion of the persistent memory 74 is used to store programs, executable code, and data.

In some embodiments, one of the inputs is from a personal activity monitor 290. The personal activity monitor 290 utilizes sensors (e.g. heart rate) to determine when a wearer is asleep or in a particular stage/cycle of sleep.

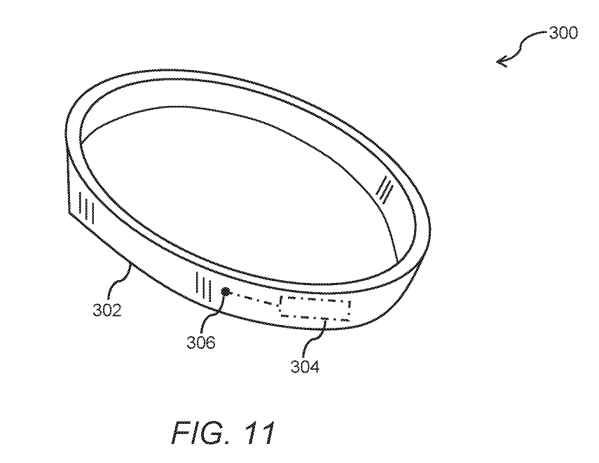

In some embodiments, local area communications with the device for delivery of a material by inhalation 10 are performed using a wireless transmitter or transceiver such as a Bluetooth radio transceiver 94, a Wi-Fi radio transceiver 96, or both. Such communication features provide data communications between the device for delivery of a material by inhalation 10 and external devices for control/configuration and for communicating environmental/user parameters. For example, in some embodiments, the device for delivery of a material by inhalation 10 receives signals indicating that a user is asleep or in a particular stage/cycle of sleep by way of the Bluetooth radio transceiver 94, for example, from a personal fitness device 300 (see FIG. 11).

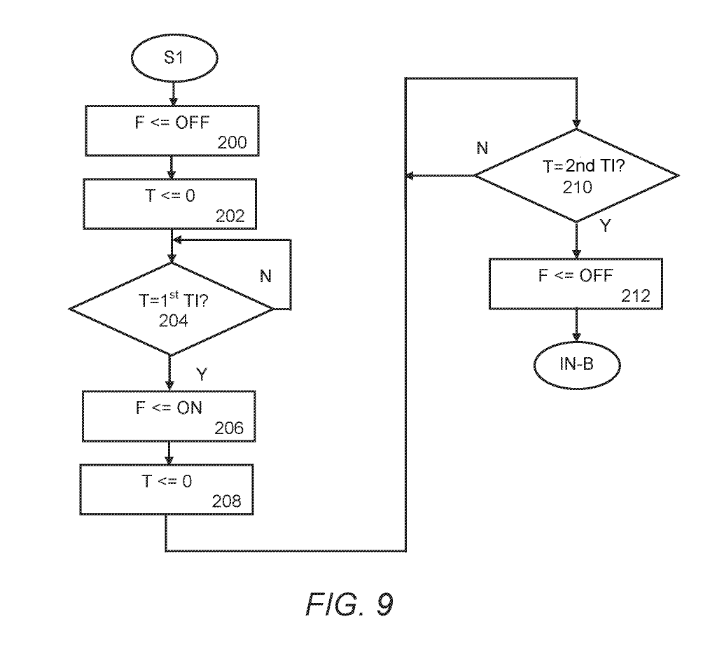

Referring to FIG. 9, a first program flow of the processor-based device for delivery of a material by inhalation 10 is shown. In this exemplary program flow, after the processor-based device for delivery of a material by inhalation 10 initializes (e.g. first switch 48 is moved to the “on” position), the first step is to turn off 200 the fan 24. A timer is reset 202 then a loop 204 runs until the timer reaches a first threshold (e.g. until 30 minutes pass). Once the timer reaches the first threshold, T1, the fan 24 is started 206 and the timer is again reset 208. As discussed previously, it is fully anticipated that in some embodiments, the program ramps power to the fan 24 to gradually start the fan 24 or to run the fan 24 at a slower rate while the payload (material) is strong, then at a faster rate as the payload wanes.

Now the fan 24 operates, emitting the material for inhalation by the user until the timer reaches a second threshold, T2 (e.g. until 2 hours pass), at which time the fan 24 is shut off 212. As with any such program, it is well known to provide programmable delays in many ways, for example utilizing a timer or looping through a set of instructions that consume a predetermined amount of time per loop. The timing and sequencing shown in FIG. 9 are examples, as other timing and sequencing are fully anticipated resulting in the same or similar operation.

Note that, in some embodiments having a second switch 46 (e.g. mode switch), the mode switch is read and if the second switch 46 is in the “instant-on” position, the first delay (loop 204) is not taken and the fan 24 is turned on immediately. Further, it is anticipated that a speed control is available to programmatically control a speed of the fan 24. Further, in some embodiments, a reader is provided, coupled to an input 92 of the device for delivery of a material by inhalation 10 providing an indication of a type of delivery cartridge 50 that has been inserted and altering the programming to deliver the payload, as discussed previously.

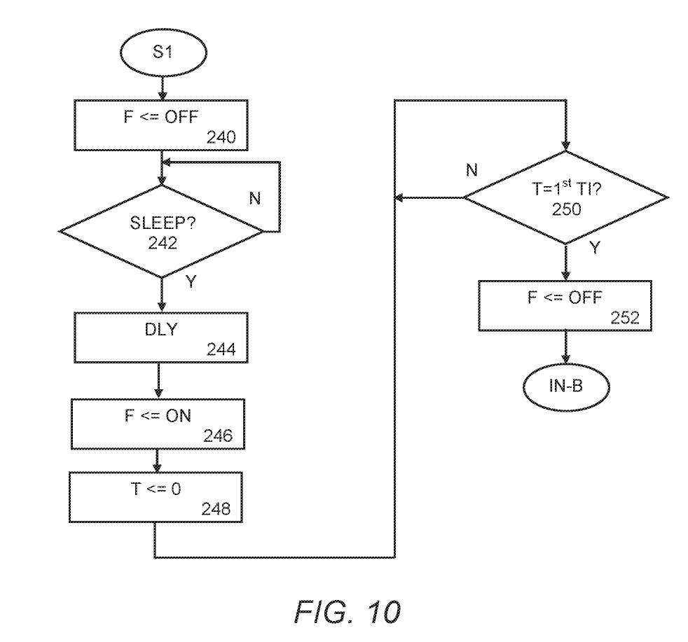

Referring to FIG. 10, a second program flow of the processor-based device for delivery of a material by inhalation 10 is shown. In this exemplary program flow, after the processor-based device for delivery of a material by inhalation 10 initializes (e.g. first switch 48—power—is moved to the “on” position), the first step is to turn off 200 the fan 24. Now, it is determined if the user is asleep or in a particular stage/cycle of sleep 242, for example, by communicating with a personal fitness device 300 (see FIG. 11) through one of the wireless interfaces 94/96 or by direct reading from a personal activity monitor 290. Once it is determined that the user is asleep or in a particular stage/cycle of sleep 242, in some embodiments, a delay 244 is taken (e.g. delay until 5 minutes pass). This optional delay 244 allows the user to be asleep for a short interval before the fan 24 is started 246. Once the fan 24 is started 246, a timer is reset 248. Now the fan 24 operates, emitting aroma therapy until the timer reaches a second threshold, TI (e.g. until 2 hours pass), at which time the fan 24 is shut off 250. As with any such program, it is well known to provide programmable delays in many ways, for example utilizing a timer or looping through a set of instructions that consume a predetermined amount of time per loop. The timing and sequencing shown in FIG. 10 are examples, as other timing and sequencing are fully anticipated resulting in the same or similar operation.

Note that, in some embodiments having a second switch 46 (e.g. mode switch), the second switch 46 is read and if the second switch 46 is in the “continuous” position, the fan 24 remains operational until the user operates the first switch 48 (power switch).

Again, as discussed above, various algorithms for timing and ramping of power to the fan 24 are anticipated to provide any desired or needed rate of delivery of the payload, over time, for the delivery of the payload for inhalation by the user.

Referring to FIG. 11, a perspective view of a personal fitness device 300 is shown. In this example of a personal fitness device 300, a bracelet band 302 encloses the personal fitness device 300. The bracelet band 302 is fabricated of a material such as silicone or fluoroelastomer. The bracelet band 302 contains logic and sensors 304 for detecting various biological and activity parameters of a wearer, such as heart rate, body temperature, footsteps, etc. In this example, the power source is not shown for clarity reasons. In this embodiment, the personal fitness device 300 transmits data periodically using an internal antenna 306, for example, utilizing Bluetooth standard. In some embodiments, the device for delivery of a material by inhalation 10 receives signals from the internal antenna 306 of the personal fitness device 300 for understanding sleep patterns of the wearer/user.

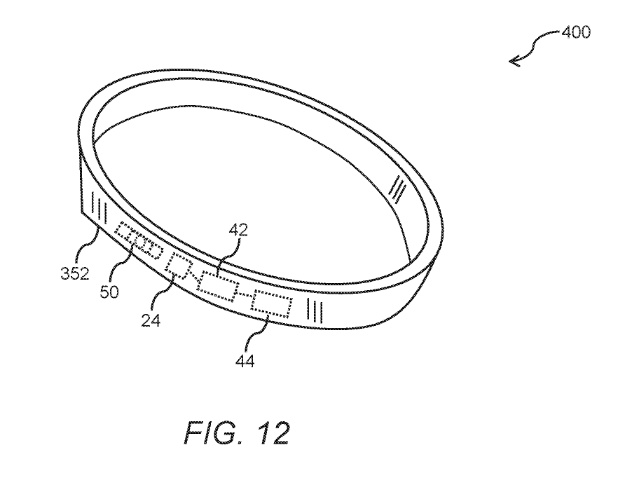

Referring to FIG. 12, a perspective view of the device for delivery of a material by inhalation 10 integrated into a wristband 352 is shown. In this embodiment, the power source 44 and control circuit 42 are integrated into a wearable device 400, in this case, a wristband 352. The fan 24 and receiver for the delivery cartridge 50 are also integrated into the wristband 352, as it is anticipated that the delivery cartridge 50 is interchangeable for replacement as needed.

In some embodiments, the device for delivery of a material by inhalation 10 is integrated into a wristband 352 includes sensors (not shown) operatively coupled to the control circuit 42. These sensors detect biological conditions and movements of a wearer of the wristband 352 (e.g. heart rate, body temperature, steps taken, motion . . . ) to determine the optimal time to emit the payload for inhalation by the user.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A system for delivery of a material for inhalation, the system comprising:
a delivery cartridge having contained there within a breakable container, the breakable container having there within a payload; and
means for flowing a gas through the delivery cartridge;
wherein after the breakable container is broken, the payload mixes with the gas for delivery to a user through inhalation.

2. The system of claim 1, wherein the system for delivery of a material for inhalation is worn by the user.

3. The system of claim 1, wherein the means for flowing the gas through the delivery cartridge includes a fan that draws the gas in through one or more vents, through the delivery cartridge, and out a grill in a direction of the user for inhalation by the user.

4. The system of claim 1, wherein the means for flowing the gas through the delivery cartridge operates at a first delay after the system for delivery of a material for inhalation is turned on.

5. The system of claim 1, wherein the means for flowing the gas through the delivery cartridge operates at a first delay after the system for delivery of a material for inhalation is turned on and until a second delay occurs, at which time the means for flowing the gas through the delivery cartridge is stopped.

6. The system of claim 1, wherein the payload is an essential oil.

7. The system of claim 1, wherein the means for flowing the gas through the delivery cartridge comprises a fan.

8. The system of claim 1, wherein the means for flowing the gas through the delivery cartridge operates after the system for delivery of a material for inhalation detects that the user is asleep.

9. The system of claim 8, wherein the system for delivery of a material for inhalation detects that the user is asleep by reading data from an activity monitor.

10. The system of claim 8, wherein the system for delivery of a material for inhalation detects that the user is asleep by receiving sleep indications over a wireless interface from a personal fitness device.

11. A method of delivering a material through inhalation, the method comprising:
flowing a gas through a delivery cartridge after having broken a breakable container within the delivery cartridge, the breakable container having there within the material;
flowing the gas towards a user for delivery of the material to the user for inhaling by the user.

12. The method of claim 11, wherein the material is selected from the group comprising an essential oil, a medicinal liquid, a perfume, THC mixed with alcohol.

13. The method of claim 11, wherein the step of flowing is controlled by a power switch and the step of flowing begins in response to turning the power switch to an on position.

14. The method of claim 11, wherein the step of flowing is controlled by a power switch and in response to turning the power switch to an on position, delaying for a first time period and then beginning the step of flowing.

15. The method of claim 14, wherein after beginning the step of flowing, delaying for a second time period and then stopping of the step of flowing of the gas through the delivery cartridge.

16. An inhalation delivery system comprising:
a housing for wearing by a user;
a delivery cartridge having contained there within at least one breakable container, each of the at least one breakable container having there within a material;
a receiver for holding the delivery cartridge;
a fan interfaced to the receiver for flowing a gas through the delivery cartridge;
vents for receiving the gas from outside of the housing;
a grill for emitting the gas containing the material;
wherein after the breakable container is broken, the material mixes with the gas as the gas flows through the delivery cartridge for inhalation by a user.

17. The inhalation delivery system of claim 16, further comprising a control circuit that operates the fan after a first delay after the inhalation delivery system is turned on.

18. The inhalation delivery system of claim 17, wherein the control circuit operates the fan until a second delay occurs, at which time the fan is stopped.

19. The inhalation delivery system of claim 16, further comprising a control circuit that operates the fan after the inhalation delivery system is turned on and after detecting that the user is asleep.

20. The inhalation delivery system of claim 16, wherein the material is a liquid selected from the group comprising an essential oil, a medicinal liquid, a solution containing THC, and a perfume.
